(19) United States
(12) Patent Application Publication
(10) Pub. No.: US 2004/0155201 A1
(43) Pub. Date: Aug. 12, 2004

(54) Toothbrush sanitizer

(75) Inventors: Bruce M. Russell, Howell, NJ (US); Robert Moskovich, East Brunswick, NJ (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 10/679,145

(22) Filed: Oct. 3, 2003

Related U.S. Application Data
(60) Provisional application No. 60/416,052, filed on Oct. 4, 2002.

(51) Int. Cl. A61L 2/10

(52) U.S. Cl. 250/455.11; 422/24

(57) ABSTRACT

A sanitizing device for personal hygiene articles, particularly toothbrushes, is disclosed. A Xenon bulb or other source of sterilizing ultraviolet light is arranged in close proximity to the article to be sterilized. The light is powered by a battery which reduces the size and increases the portability of the sanitizing device. A magnifying glass or Fresnel lens can be arranged within the device between the light and article to be sanitized to focus the light on, and increase heat around, the article to be sanitized.

### CLAIM FOR PRIORITY

The present application claims priority of U.S. Provisional Patent Application Serial No. 60/416,052, filed Oct. 4, 2002, the disclosure of which being incorporated by reference herein in their entireties.

### BACKGROUND OF THE INVENTION

Toothbrushes, especially when wet, are a natural haven for bacterial growth. Thus, there is a need for means to sanitize toothbrushes and other oral hygiene products between uses. Various attempts to meet that goal have been tried.

One approach is described in U.S. Pat. No. 4,740,7096 which illustrates a sanitizer for bathroom articles, including toothbrushes. That device includes a germicidal lamp and a domed floor which drains water from the articles toward the lower periphery of the sanitizer housing and then through drainage holes in that housing. The germicidal lamp can be a circular flourescent ultraviolet (UV) light or other known germicidal lamp (see column 5, lines 1-5). See also U.S. Pat. No. 4,625,119 containing a similar disclosure.

U.S. Pat. No. 4,803,364 discloses an ultraviolet radiation source such as a UV lamp to sterilize and dry toothbrush bristles. The UV lamp is housed in a device that promotes removal of moisture from around the toothbrush, thereby minimizing re-condensation of bacteria laden moisture on the toothbrush. (See column 1, lines 48-54).

Use of disinfectants and ultrasonic radiation to sterilize medical and dental instruments is disclosed in U.S. Pat. No. 4,448,650. UV light and heat may also be used for added sterilization effect.

U.S. Pat. No. 5,023,460 describes a toothbrush sanitizer in which toothbrushes are arrayed about the periphery of a UV light. Behind each toothbrush is a mirror-like surface that reflects UV light and its sanitizing effect, on all surfaces of the toothbrush (column 2, lines 10-18).

In U.S. Pat. No. 5,614,151 a columnar ultraviolet bulb surrounds the object to be sanitized, thereby exposing all surfaces of the object to the sanitizing effect of the UV light without shadowing. Air introduced into the columnar enclosure is excited to produce ozone which has an additional sanitizing effect.

De-moisturizing toothbrushes, and other moisture sensitive devices like hearing aids, is the goal of U.S. Pat. No. 5,852,879. Air within the apparatus containing the toothbrush is circulated through a desiccant to de-moisturize those items.

Heated air and sprayed anti-microbial disinfectant are used to sanitize toothbrushes according to the teachings of U.S. Pat. No. 6,171,559B1.

A commercial toothbrush sanitation device which generates ozone to sanitize toothbrushes is sold by Cyclopss Corporation and is described at www.cyclopss.com.

Each of these devices operates on household current and thus is not readily transportable. In addition, each has some limitation on its effectiveness as a sanitation device.

### SUMMARY OF THE INVENTION

The sanitizing system of this invention provides a unique combination of reduced size, ready transportability, battery operation and effective sanitation of objects such as toothbrushes. This new sanitizing system utilizes light emitting diodes that emit light in the long wave ultraviolet range (350-400 mm) referred to as UV-C radiation. Although ultraviolet light is somewhat effective at shorter wavelengths (100-300 mm), some studies indicate that the sanitizing effect of the shorter wavelength (UV-A emission) is not as effective as the longer wavelength (UV-C) emissions. The sanitizing effect of UV-A emissions can, however, be enhanced by heating the air through which the emissions travel. Heat by itself also has a sanitizing effect, if for no other reason than it helps to de-moisturize articles. This removes the breeding ground for bacteria.

To get the beneficial effect of UV emissions and light, this invention utilizes a Xenon bulb in close proximity to the article to be sanitized such as a toothbrush head. The desired heating can be accomplished by transmitting the light from this bulb through a Fresnel lens or a magnifying glass. The generated heat is also useful in killing bacteria. The heating can also be accomplished using infrared emitting diodes.

To promote transportability and reduce dependence on household current, the present invention may be battery powered. Dry cell batteries or wafer batteries can be used to power the UV emitting diodes or the infrared emitting diodes. In another embodiment, these batteries can be recharged using solar power.

When used to sanitize a toothbrush, these power sources and light emitting diode(s) are preferably contained in a toothbrush holder where the diodes are located in close proximity to the toothbrush head. This facilitates the heating of the toothbrush cleaning elements and the air surrounding those elements. It also allows the most intense emissions to be directed at the portion of the toothbrush most likely to be bacteria laden.

### BRIEF DESCRIPTION OF THE DRAWINGS

FIG. 1 is a cross-sectional view in elevation showing a toothbrush head being sanitized in one embodiment of this invention.

FIG. 2 is a cross-sectional view in elevation showing a power and manual toothbrush placed in a sanitation device of this invention.

FIG. 3 is a cross sectional view showing a toothbrush in a fully enclosed sanitation device of this invention.

### DETAILED DESCRIPTION OF THE INVENTION

The sanitizing device of this invention is preferably an enclosed case surrounding the article to be sanitized. For purposes of illustration, the drawings of this application show the sanitation of a toothbrush, however, the principles of the invention can be applied to other household or personal articles that need to be sanitized.

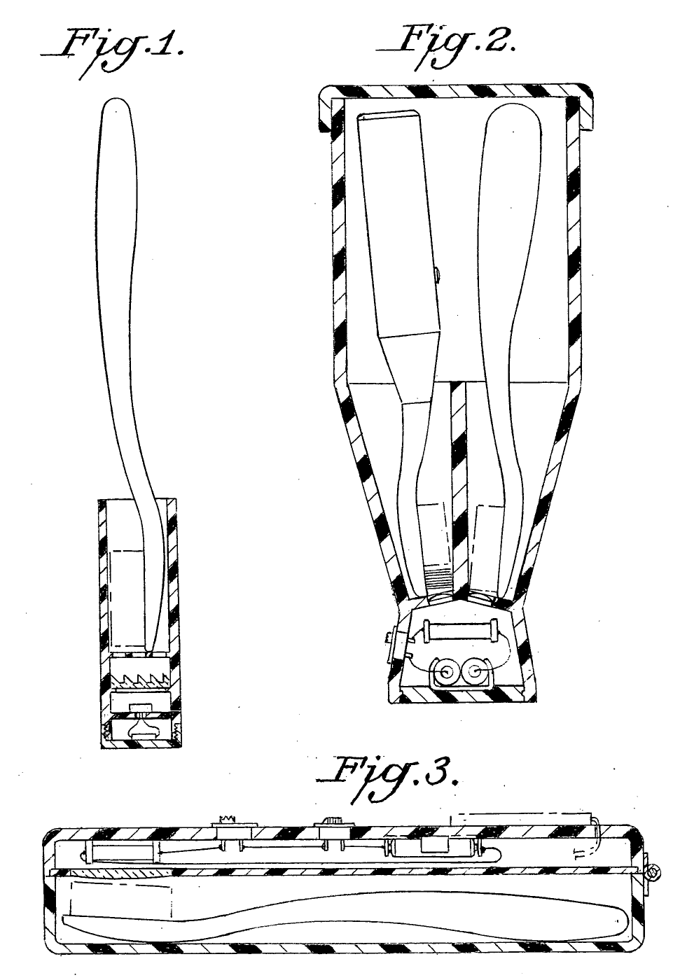

As illustrated in FIG. 1, the sanitizing device 10 of this invention surrounds the head 14 of manual toothbrush 12 on three sides. The device 10 contains a battery 16 which powers a light emitting device 18. A Fresnel lens 20 is arrayed between the LED 18 and the toothbrush head 14 to concentrate the light on the toothbrush head 14 as well as heat the air surrounding it. Alternatively, a magnifying glass can be used to achieve these goals. The toothbrush head 14 is separated from the LED 18 and lens 20 by a perforated platform 22. The openings in platform 22 support the toothbrush 12 above the light and heat sources and allow bacterial laden moisture on the toothbrush head 14 to drain out of the area immediately surrounding the toothbrush head 14.

The light emitting device 18 is preferably a Xenon bulb which emits a wavelength characteristic of sunlight. In addition, the Xenon lamp will become hot and this higher temperature will be beneficial in sanitizing the toothbrush head. The Fresnel or magnifying lens 20 will concentrate the rays emitted and generate more head that is useful in killing bacteria.

FIG. 2 illustrates an embodiment of the invention in which the sanitizing device 10 fully surrounds the device(s) to be sanitized, in this case, manual toothbrush 12 and powered toothbrush 12A. In this embodiment, magnifying lenses 20 direct the light from light emitting device 18 onto toothbrush heads 14. The LED 18 is connected to battery power 16 (shown as two AA batteries) through an on-off switch 24. The sanitizing device 10 is appropriately shaped to accept the devices to be sanitized. As illustrated in FIG. 2, the sanitizing device 10 includes an enclosure cap 26. Enclosing the sanitation device 10 increases the heating effect of the light emitting device 18 as amplified by the lens 20. As with the device shown in FIG. 1, the toothbrush heads 14 in FIG. 2 are arranged in close proximity to the light 18 to take full advantage of the sterilizing emissions therefrom.

The sanitizing device 10 shown in FIG. 3 includes a two part hinged case. The upper portion 28 of the device 10 is swingably attached to the lower portion 30 by a hinge 32. The upper portion preferably contains a base 29 which fully or partially encloses the functional elements of the sanitizing device 10. These elements, battery 16, light emitting device 18, lens 20 and on-off switch 24 operate in the manner previously described. Also shown in this embodiment (FIG. 3) is a timer 25 that can be set for a period of time found most effective in sanitizing articles. Another feature shown (in phantom) in this embodiment is solar cell 34 which is electrically connected to batteries 16 to recharge them. The FIG. 3 embodiment of the sanitizing device 10 can be used by rotating upper portion 28 of the device 10 about hinge 32, placing the toothbrush 12 in the lower portion 30 of the sanitizing device 10 and closing the upper portion 28 onto the lower portion 30. The switch 24 is then turned on and the timer 25 set for an appropriate period to sanitize toothbrush 12.

What is claimed is:

1. A portable, battery operated sanitizing device for a toothbrush comprising an enclosure about all or part of a toothbrush head, a light emitting device within the enclosure and in close proximity to the toothbrush head capable of emitting light in the ultraviolet range and a battery within or associated with the device for powering the light emitting device.

2. The sanitizing device of claim 1 wherein the light emitting device is a Xenon bulb.

3. The sanitizing device of claim 1 wherein the light emitting device is capable of emitting light in the long wave ultraviolet range of 350-400 mm.

4. The sanitizing device of claim 1 wherein a solar panel is associated with the device to recharge the battery.

5. The sanitizing device of claim 1 wherein a magnifying glass or Fresnel lens is arranged between the light emitting device and the toothbrush head.

6. The sanitizing device of claim 1 wherein a timer is electrically connected between the battery and light.
